(19) United States
(12) Patent Application Publication
(10) Pub. No.: US 2024/0272030 A1
(43) Pub. Date: Aug. 15, 2024

(54) Testing apparatus for smart wearable devices

(71) Applicant: Plume Design, Inc., Palo Alto, CA (US)

(72) Inventors: Hao-Hsiu Huang, Taipei City (TW); Chun Hung Liu, Taipei City (TW); Zhou Cheng, Taipei City (TW); Ming-Tsung Su, Taipei City (TW)

(21) Appl. No.: 18/109,778

(22) Filed: Feb. 14, 2023

(51) Int. Cl. G01M 3/02; G06F 1/16

(52) U.S. Cl. CPC G01M 3/02; G06F 1/163; H05K 5/06

(57) ABSTRACT

A testing apparatus includes a bottom tank comprising a plurality of coupling connectors; and a test shaft that includes a member that is rotatably connected to the plurality of coupling connectors, a plurality of perpendicular members that extend out from the member, and a plurality of device under test (DUT) members connected to the plurality of perpendicular members, wherein the plurality of DUT members each include a plurality of posts each to support a DUT.

### FIELD OF THE DISCLOSURE

The present disclosure relates generally to a test apparatus. More particularly, the present disclosure relates to systems and methods for a testing apparatus for smart wearable devices.

### BACKGROUND OF THE DISCLOSURE

Smart wearable devices can include, for example, smart rings, smart watches, fitness bands, fitness trackers, pedometers, head mounted displays, smart glasses, and the like. One function of smart devices is to track motion, e.g., steps, swimming distance, running pace, and the like. An example smart wearable device is a smart ring, such as described in commonly-assigned U.S. patent application Ser. No. 17/714,220, filed Apr. 6, 2022, and entitled “Smart ring for use with a user device and Wi-Fi network,” the contents of which are incorporated by reference. One aspect of the smart wearable device is being waterproof, see, e.g., commonly-assigned U.S. patent application Ser. No. 18/105,441, filed Feb. 3, 2023, and entitled “Edge rib and laser etching treatment to improve waterproofing of smart ring,” the contents of which are incorporated by reference. There is a need to test both the ability of smart wearable devices to accurately track motion (steps, swimming laps, etc.) and to remain waterproof and the like.

### BRIEF SUMMARY OF THE DISCLOSURE

The present disclosure relates to systems and methods for a testing apparatus for smart wearable devices. The testing apparatus is configured to test motion and optionally waterproofing of smart wearable devices. In an embodiment, the smart wearable devices can be smart rings. The motion can be related to swimming and the testing apparatus can include a water tank to test the waterproofing of the smart wearable devices. In an embodiment, the testing apparatus can be configured to support a plurality of smart wearable devices for simultaneous testing, such that the corresponding motion can be determined by comparing the plurality of smart wearable devices. The testing apparatus can be used to test prototype smart wearable devices, as well for factory testing, i.e., any type of testing is contemplated.

In an embodiment, a testing apparatus includes a bottom tank comprising a plurality of coupling connectors; and a test shaft that includes a member that is rotatably connected to the plurality of coupling connectors, a plurality of perpendicular members that extend out from the member, and a plurality of device under test (DUT) members connected to the plurality of perpendicular members, wherein the plurality of DUT members each include a plurality of posts each to support a DUT.

The DUT can be a smart ring or a smart watch. The testing apparatus can further include a removable top cover located on the bottom tank. The testing apparatus can further include a motor connected to the member and configured to rotate the member about the plurality of coupling connectors.

The testing apparatus can further include a motor connected to the member and configured to rotate the member about the plurality of coupling connectors in a 360-degree motion to simulate swimming motion where the posts simulate a swimmer's hands and the plurality of perpendicular members simulate the swimmer's arms. The testing apparatus can further include a motor connected to the member and configured to rotate the member about the plurality of coupling connectors in a 180-degree motion back and forth to simulate a walking or running motion where the posts simulate a wearer's hands and the plurality of perpendicular members simulate the wearer's arms.

The testing apparatus can further include water in the bottom tank for simulating swimming. The bottom tank can include water for simulating swimming and air for simulating walking or running. The testing apparatus can further include a motor connected to the member and configured to rotate the member about the plurality of coupling connectors; and a control panel for controlling and configuring the motor. The plurality of perpendicular members can include a first perpendicular member and a second perpendicular member located about 180 degrees from the first perpendicular member.

The first perpendicular member can include a first DUT member and the second perpendicular member includes a second DUT member, and wherein the first DUT member and the second perpendicular member can include complementary DUTs. The complementary DUTs experience a same amount motion in a given test, such that each of the complementary DUTs should include similar metrics for the given test to be successful.

In another embodiment, a method of testing a smart wearable device includes steps of placing a plurality of smart wearable devices on a test shaft that includes (1) a member that is rotatably connected to a plurality of coupling connectors on a bottom tank, (2) a plurality of perpendicular members that extend out from the member, and (3) a plurality of device under test (DUT) members connected to the plurality of perpendicular members, wherein the plurality of DUT members each include a plurality of posts each to support a smart wearable device of the plurality of smart wearable devices; and rotating the test shaft in the bottom tank that includes the plurality of coupling connectors.

The rotating can be in a 360-degree motion to simulate swimming motion where the posts simulate a swimmer's hands and the plurality of perpendicular members simulate the swimmer's arms. The rotating can be in a 180-degree motion back and forth to simulate a walking or running motion where the posts simulate a wearer's hands and the plurality of perpendicular members simulate the wearer's arm. The bottom tank can include water for simulating swimming and air for simulating walking or running.

The plurality of perpendicular members can include a first perpendicular member and a second perpendicular member located about 180 degrees from the first perpendicular member, wherein the first perpendicular member includes a first DUT member and the second perpendicular member includes a second DUT member, and wherein the first DUT member and the second perpendicular member include complementary DUTs, and wherein the method can further include checking if the complementary DUTs experience a same amount motion in a given test, such that each of the complementary DUTs should include similar metrics for the given test to be successful.

The bottom tank can include water for simulating swimming, and the method can further include checking the plurality of smart wearable devices to determine whether they remain waterproof after a given test. The smart wearable device can be one or more of a smart ring and a smart watch.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a perspective view of a testing apparatus with a top cover included thereon.

FIG. 2 is a perspective view of the testing apparatus with the top cover shown transparently.

FIG. 3 is another perspective view of the testing apparatus with the top cover shown transparently.

FIG. 4 is the perspective view of FIG. 3 of the testing apparatus with the top cover and a bottom tank shown transparently.

FIG. 5 is a perspective view of a test shaft.

FIG. 6 is a close-up view of the perspective view of FIG. 5 of the test shaft.

FIG. 7 is a further cloud-up view of the perspective view of FIG. 6 illustrating a single ring on the test shaft.

FIG. 8 is a side view of the testing apparatus illustrating motion of the test shaft to simulate swimming.

FIG. 9 is a flowchart of a process of testing a smart wearable device.

### DETAILED DESCRIPTION OF THE DISCLOSURE

Again, the present disclosure relates to systems and methods for a testing apparatus for smart wearable devices. The testing apparatus is configured to test motion and optionally waterproofing of smart wearable devices. In an embodiment, the smart wearable devices can be smart rings. The motion can be related to swimming and the testing apparatus can include a water tank to test the waterproofing of the smart wearable devices. In an embodiment, the testing apparatus can be configured to support a plurality of smart wearable devices for simultaneous testing, such that the corresponding motion can be determined by comparing the plurality of smart wearable devices. The testing apparatus can be used to test prototype smart wearable devices, as well for factory testing, i.e., any type of testing is contemplated.

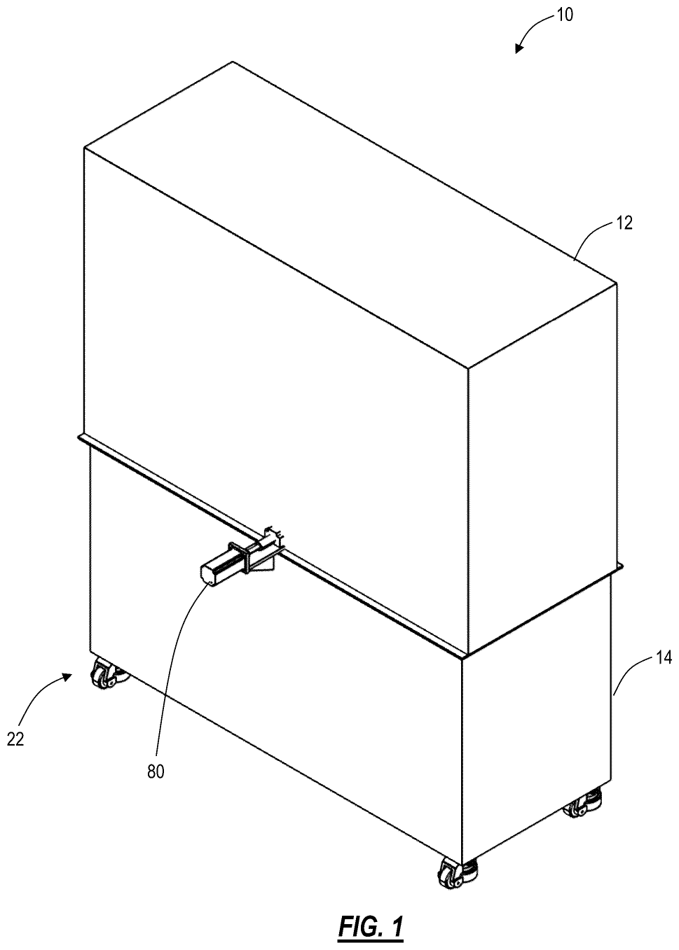

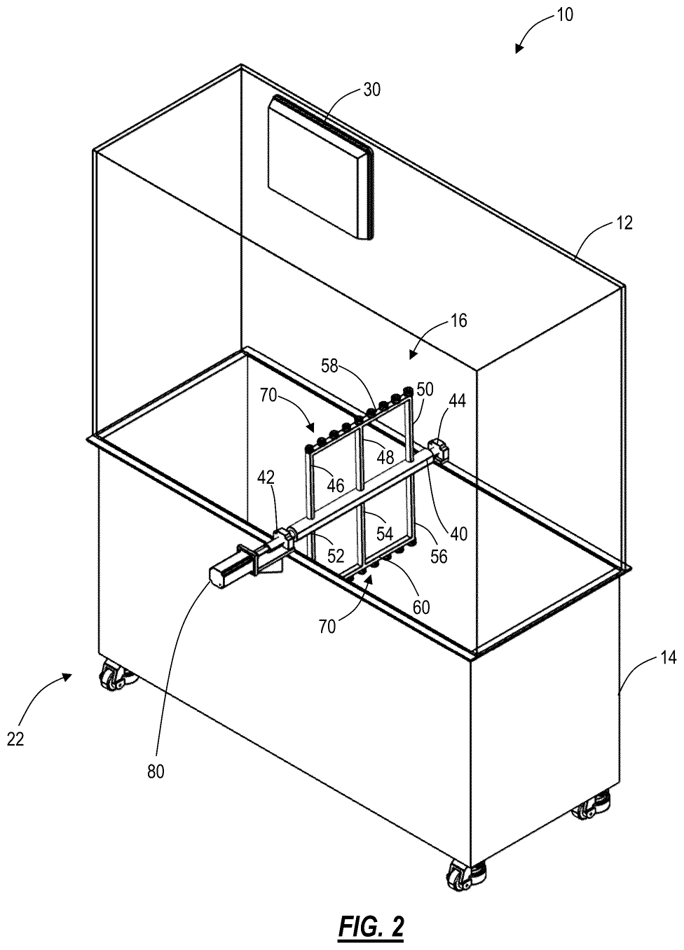

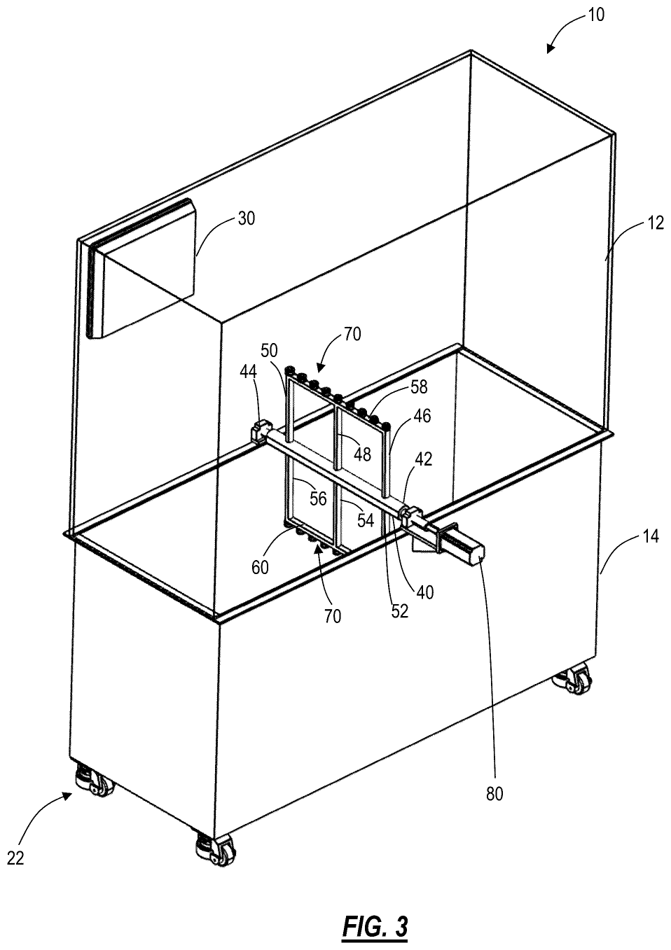

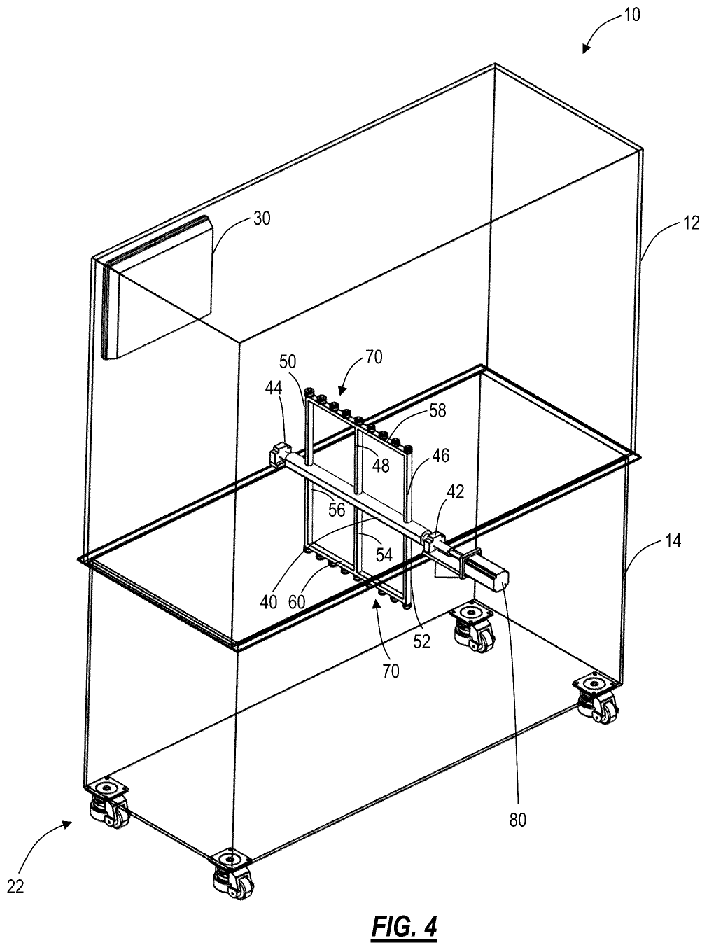

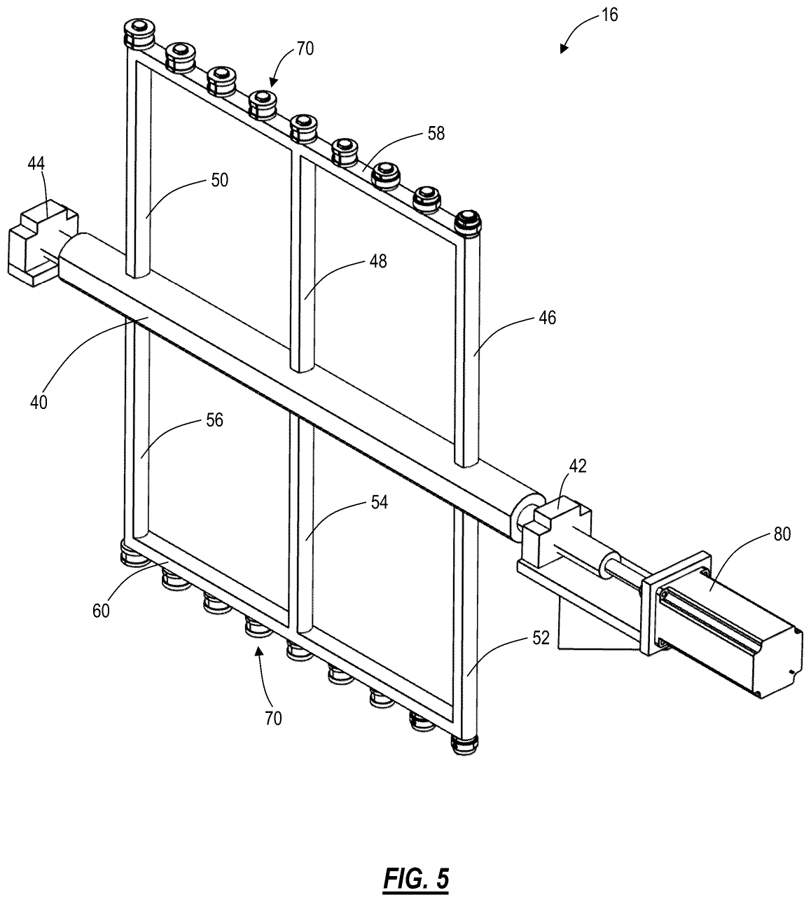

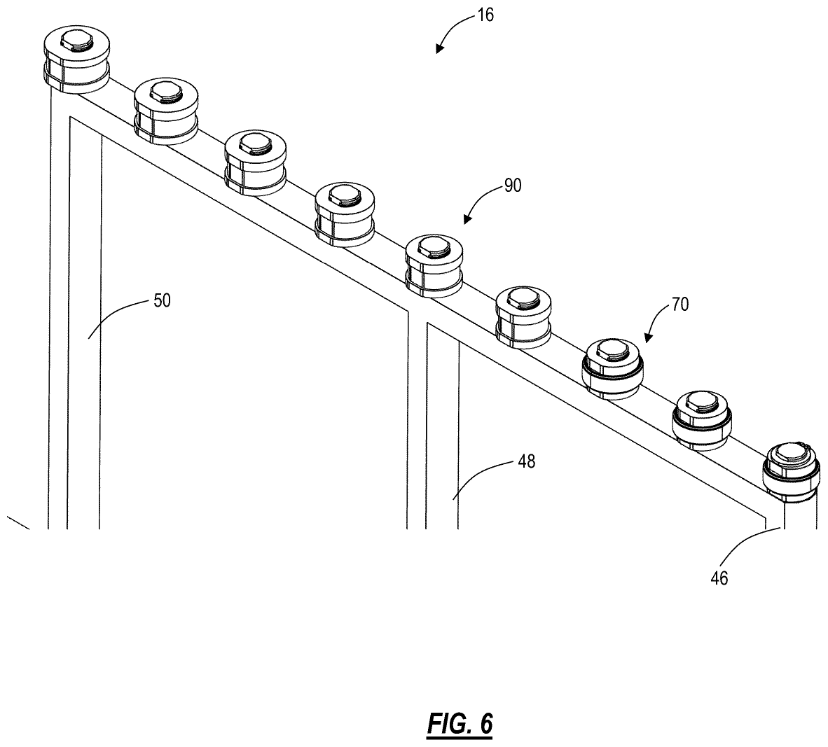

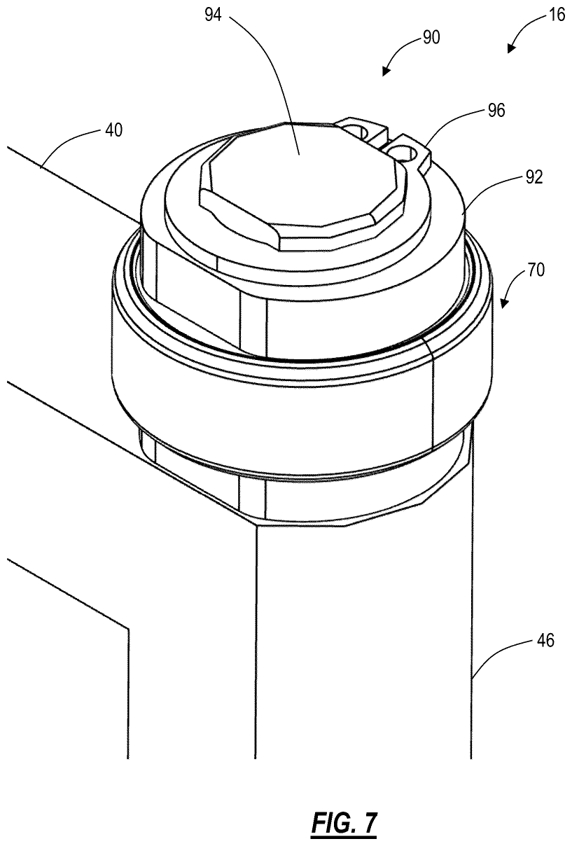

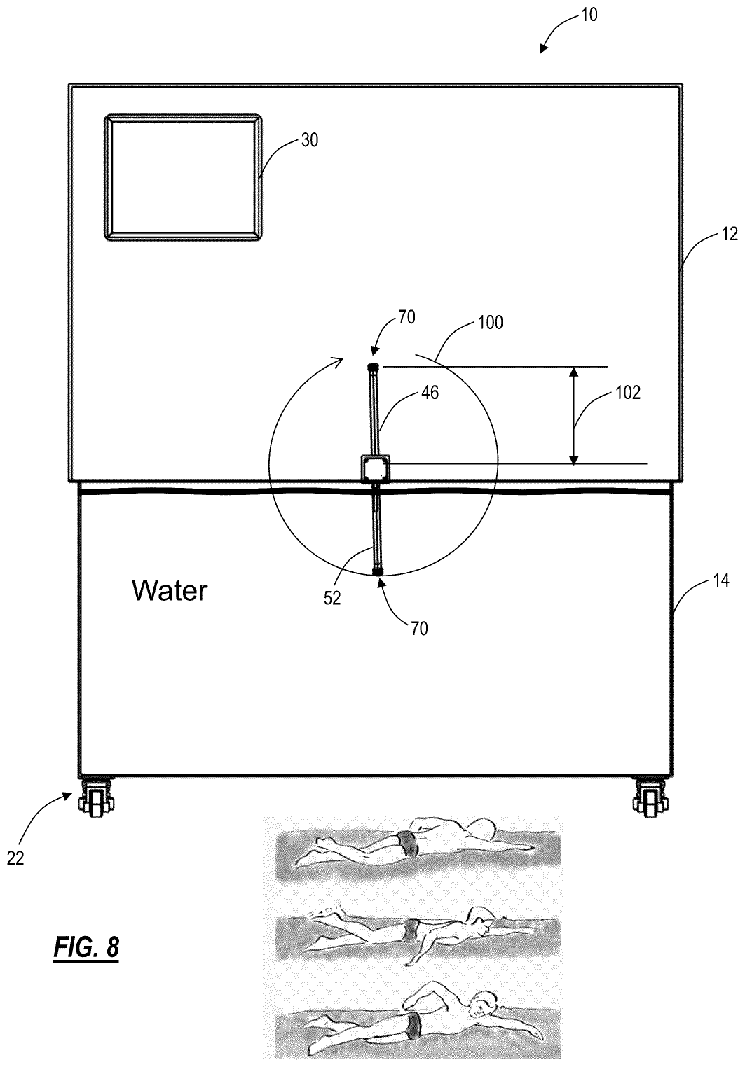

FIG. 1 is a perspective view of a testing apparatus with a top cover 12 included thereon. FIG. 2 is a perspective view of the testing apparatus with the top cover 12 shown transparently. FIG. 3 is another perspective view of the testing apparatus with the top cover 12 shown transparently. FIG. 4 is the perspective view of FIG. 3 of the testing apparatus with the top cover 12 and a bottom tank 14 shown transparently. FIG. 5 is a perspective view of a test shaft 16. FIG. 6 is a close-up view of the perspective view of FIG. 5 of the test shaft 16. FIG. 7 is a further cloud-up view of the perspective view of FIG. 6 illustrating a single ring 20 on the test shaft 16. FIG. 8 is a side view of the testing apparatus 10 illustrating motion of the test shaft 16 to simulate swimming.

The descriptions herein are directed to the testing apparatus 10 for testing multiple smart rings 20 simultaneously for waterproofing and swimming tracking. Those skilled in the art will recognize the testing apparatus 10 can be used for other types of smart wearable devices, e.g., smart watches, fitness bands, fitness trackers, pedometers, head mounted displays, smart glasses, and the like. Further, those skilled in the art will recognize the testing apparatus 10 can be used for other motions instead of swimming, such as walking, running, etc.

The testing apparatus 10 includes a top cover 12 that is removable, and a bottom tank 14. The bottom tank 14 can have casters 22 for supporting the testing apparatus 10 and for moving the testing apparatus. In an embodiment, the top cover 12 and/or the bottom tank 14 can be clear, i.e., made of a transparent material—glass, plastic, etc. To simulate swimming, the bottom tank 14 is filled with water. Note, to simulate walking or running, the bottom tank 14 can just have air.

The testing apparatus 10 can have a control panel 30, such as on the top cover 12. The control panel 30 can include a touch screen, buttons, visual indicators, etc., and is used to set the configuration of a given test to be run in the testing apparatus 10. The control panel 30 can include settings for an automated test run, e.g., time, motion (e.g., swimming, running, walking, etc.), number of devices under test (DUT), type of DUT (e.g., ring, watch, etc.), content of the bottom tank 14 (e.g., water, air, etc.), speed of motion, etc. The control panel 30 can also include input/output mechanisms such as Wi-Fi, Bluetooth, wired ports (e.g., USB, Ethernet), and the like. The control panel 30 can operate with an application on a mobile device, via a webpage on a computer, and the like. The control panel 30 can receive configuration via the input/output mechanisms as well as provide details of any test runs.

In addition, the control panel 30 can include mobile apps executed thereon to communicate with the DUTs. For example, assume there are 18 DUTs, namely smart rings 20, then the control panel 30 can operate 18 different instances of a mobile app for communicating with each of the smart rings 20. In addition, the control panel 30 can obtain desired metrics from each smart ring 20 for comparison. This is described in further detail herein, namely one important aspect of any test run is to ensure the given rings report the same or similar metrics, e.g., steps, swimming laps, distance, etc.

Operationally, the test shaft 16 is configured to simulate motion while the DUTs are disposed on the test shaft 16. That is the test shaft 16 can rotate (swimming motion) or swing (walking or running motion) to simulate motion. Details are now described for the test shaft 16.

The test shaft 16 includes a member 40 that is rotatably connected to the bottom tank 14, e.g., via coupling connectors 42, 44 on the bottom tank 14. The member 40 is configured to rotate about the coupling connectors 42, 44. For example, the member 40 can include ball bearings or the like at each end, and the ball bearings are rotatably connected to the coupling connectors 42, 44. Those skilled in the art will appreciate various mechanical coupling approaches are contemplated.

The member 40 includes one or more perpendicular members 46, 48, 50, 52, 54, 56 that are disposed, attached, or connected thereto. The example in FIGS. 1-8 illustrates three perpendicular members extending outward from the member 40, with two sets of perpendicular members at 180 degrees or so from one another. The perpendicular members 46, 48, 50 connect to a DUT member 58, and the perpendicular members 52, 54, 56 connect to a DUT member 60. There can be other numbers of perpendicular members, e.g., one to two, or more than three, as long as they physically support the DUT members 58, 60. The objective of the perpendicular members 46, 48, 50, 52, 54, 56 is to extend the DUTs from the member 40, to simulate arms, i.e., the perpendicular members 46, 48, 50, 52, 54, 56 have a given length that extends from the member 40.

The DUT members 58, 60 are configured to support various DUTs 70. In the example of FIGS. 1-8, the DUTs 70 are smart rings 20. Those skilled in the art will recognize other DUTs 70 are also contemplated, e.g., smart watches, etc. Also, the member 40 is connected to a motor 80, e.g., a servo motor that is configured to rotate the member 40, thereby rotating the DUT members 58, 60, to similar motion. The motor 80 can be configured by the control panel 30.

The DUT members 58, 60 include a plurality of posts 90, each for supporting a DUT 70, i.e., smart ring 20. The posts 90 are dimensioned according to the size of the DUT 70. For example, for the smart rings 20, the posts 90 are sized similar to human fingers. For smart watches, the posts 90 can be sized similar to human wrists, etc.

The posts 90 include a rubber sleeve 92 surrounding a rigid post 94. There can be a clasp 96 which can be tightened around the post 90 to fix the rubber sleeve 92 on the rigid post 94. The DUT 70, i.e., the smart ring 20, can be placed over the rubber sleeve 92.

In an embodiment, the DUT members 58, 60 include complementary DUTs 70, spaced 180 degrees or so apart. This enables testing in the sense that two complementary DUTs 70 can be compared and their tracked motion, distance, etc. can be compared. This is because any motion caused by the motor 80, the member 40, etc. should be seen equal by the complementary DUTs 70.

In an embodiment, the DUT members 58, 60 each include 9 posts, for supporting a range of smart rings 20 at different sizes. As can be appreciated by one or ordinary skill in the art, the posts 90 can include variable sizes, such as based on the size of the rubber sleeve 92, to support the different size smart rings 20. For a smart watch, the posts 90 can be similarly sized as watches can include different sized bands which can be set to fixedly attach the smart watch to the post 90.

Again, the smart rings 20 can be tested for waterproofing ability, i.e., by being submerged in the bottom tank 14 having water included therein, while the test shaft 16 is used to simulate swimming. The smart rings 20 can be positioned on the posts 90 such that a photoplethysmography (PPG) sensor on the smart ring 20 touching the water first on each swing.

FIG. 8 is a diagram of the test apparatus 10 for illustrating the motion to simulate swimming. A motion 100 for swimming includes a 360-degree rotation of the member 40, thereby causing the DUT member 58, 60 to simulate a wearer using her arms to swim. The objective of a swimming test is to simulate the swimmer's posture in the water, to test the smart ring 20 against the water's impact, with the rotation speed adjusted to fit the pressure to simulate real swimming and to ensure the smart ring 20 can maintain its normal function in the test.

To that end, the perpendicular members 46, 48, 50, 52, 54, 56 can have a length 102 up to 250 mm, the motor 80 is configured to rotate the member 40 360-degrees in a clockwise motion, at variable speeds, such as between about 65 to 75 Revolutions per Minute (RPM).

The swimming test can run variable amounts of time, with variable speeds, and the like to check the smart rings 20 tolerance to water, their ability to accurately track swimming motion, and the like.

FIG. 8 illustrates the swimming test. The testing apparatus 10 can also simulate walking or running with the same equipment. As shown in FIG. 8, the swimming motion requires the member to be rotated in a 360-degree motion. For walking/running, the same motor 80 and test shaft 16 can be used, but instead of the 360-degree motion, the configuration can be configured to rotate in a pendulum motion at about 180-degrees, back and forth, to simulate movement of arms while walking or running.

The testing apparatus 10 contemplates any type of testing, whether prototypes to test initial functionality, as well as manufacturing testing where some or all DUTs are placed in the testing apparatus 10 to check functionality. As is well known in manufacturing, it is possible to spot test devices off the assembly line.

After completion of a given test, there can be several checks to ensure the test was successfully completed. For swimming, the DUTs 70 can be checked to ensure functionality after submersion, i.e., waterproof ability. For swimming, running, and walking, various metrics can be checked to ensure consistency between DUTs 70, i.e., because multiple DUTs 70 all are given the same motion, they should be consistent with respect to metrics, namely laps, steps, distance, time, etc.

Also, while illustrated with the control panel 30 and the motor 80, those skilled in the art will appreciate the testing apparatus 10 can be manual, without the motor 80, replaced by a handle where an operator can rotate the test shaft 16 manually.

In an embodiment, a testing apparatus 10 includes a bottom tank 14 with a plurality of coupling connectors 42, 44; and a test shaft 16 that includes a member 40 that is rotatably connected to the plurality of coupling connectors 42, 44, a plurality of perpendicular members 46, 48, 50, 52, 54, 56 that extend out from the member 40, and a plurality of device under test (DUT) members 58, 60 connected to the plurality of perpendicular members 46, 48, 50, 52, 54, 56, wherein the plurality of DUT members 58, 60 each include a plurality of posts 90 each to support a DUT 70.

The DUT 70 can be a smart ring or a smart watch. The testing apparatus 10 can include a removable top cover 12 located on the bottom tank 14. The testing apparatus 10 can include a motor 80 connected to the member 40 and configured to rotate the member 40 about the plurality of coupling connectors 42, 44. The testing apparatus 10 can include a motor 80 connected to the member 40 and configured to rotate the member 40 about the plurality of coupling connectors 42, 44 in a 360-degree motion to simulate swimming motion where the posts 90 simulate a swimmer's hands and the plurality of perpendicular members 46, 48, 50, 52, 54, 56 simulate the swimmer's arms. The testing apparatus 10 can include a motor 80 connected to the member 40 and configured to rotate the member 40 about the plurality of coupling connectors 42, 44 in a 180-degree motion back and forth to simulate a walking or running motion where the posts 90 simulate a wearer's hands and the plurality of perpendicular members 46, 48, 50, 52, 54, 56 simulate the wearer's arms.

The testing apparatus 10 can include water in the bottom tank 14 for simulating swimming. The bottom tank 14 can include water for simulating swimming and air for simulating walking or running. The testing apparatus 10 can include a motor 80 connected to the member 40 and configured to rotate the member 40 about the plurality of coupling connectors 42m 44; and a control panel 30 for controlling and configuring the motor 80.

The plurality of perpendicular members 46, 48, 50, 52, 54, 56 include first perpendicular members 46, 48, 50 and second perpendicular members 52, 54, 56 located about 180 degrees from the first perpendicular member. The first perpendicular member includes a first DUT member and the second perpendicular member includes a second DUT member, and wherein the first DUT member and the second perpendicular member include complementary DUTs. The complementary DUTs experience a same amount motion in a given test, such that each of the complementary DUTs should include similar metrics for the given test to be successful.

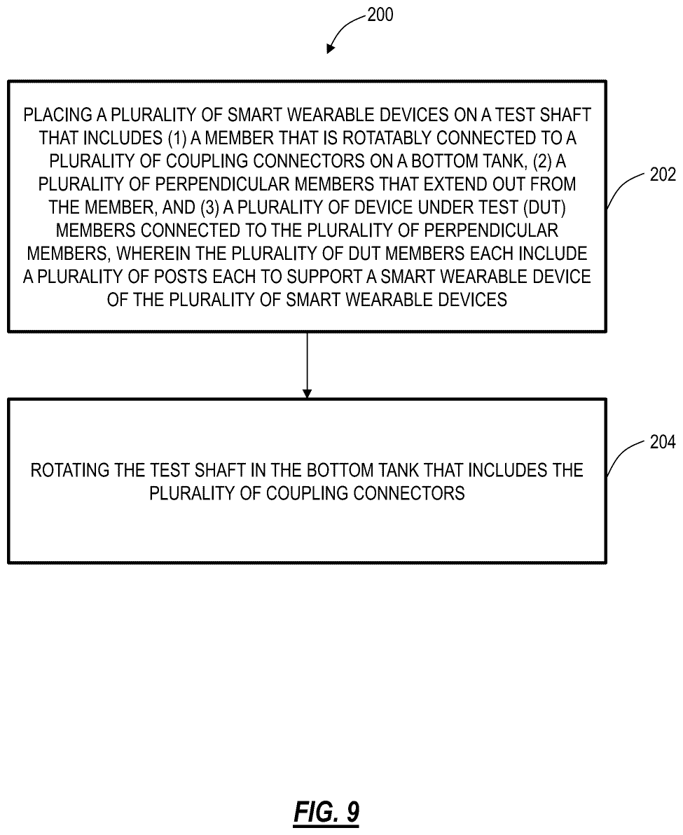

FIG. 9 is a flowchart of a process 200 of testing a smart wearable device. The process 200 includes placing a plurality of smart wearable devices on a test shaft that includes (1) a member that is rotatably connected to a plurality of coupling connectors on a bottom tank, (2) a plurality of perpendicular members that extend out from the member, and (3) a plurality of device under test (DUT) members connected to the plurality of perpendicular members, wherein the plurality of DUT members each include a plurality of posts each to support a smart wearable device of the plurality of smart wearable devices (step 202); and rotating the test shaft in the bottom tank that includes the plurality of coupling connectors (step 204).

The rotating can be in a 360-degree motion to simulate swimming motion where the posts simulate a swimmer's hands and the plurality of perpendicular members simulate the swimmer's arms. The rotating can be in a 180-degree motion back and forth to simulate a walking or running motion where the posts simulate a wearer's hands and the plurality of perpendicular members simulate the wearer's arm. The bottom tank can include water for simulating swimming and air for simulating walking or running.

The plurality of perpendicular members can include a first perpendicular member and a second perpendicular member located about 180 degrees from the first perpendicular member, wherein the first perpendicular member includes a first DUT member and the second perpendicular member includes a second DUT member, and wherein the first DUT member and the second perpendicular member include complementary DUTs, and wherein the process 200 can further include checking if the complementary DUTs experience a same amount motion in a given test, such that each of the complementary DUTs should include similar metrics for the given test to be successful.

The bottom tank can include water for simulating swimming, and the process 200 can further include checking the plurality of smart wearable devices to determine whether they remain waterproof after a given test. The smart wearable can be one or more of a smart ring and a smart watch.

### CONCLUSION

It will be appreciated that some embodiments described herein may include one or more generic or specialized processors (“one or more processors”) such as microprocessors; central processing units (CPUs); digital signal processors (DSPs): customized processors such as network processors (NPs) or network processing units (NPUs), graphics processing units (GPUs), or the like; field programmable gate arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more application-specific integrated circuits (ASICs), in which each function or some combinations of certain of the functions are implemented as custom logic or circuitry. Of course, a combination of the aforementioned approaches may be used. For some of the embodiments described herein, a corresponding device in hardware and optionally with software, firmware, and a combination thereof can be referred to as “circuitry configured or adapted to,” “logic configured or adapted to,” etc. perform a set of operations, steps, methods, processes, algorithms, functions, techniques, etc. on digital and/or analog signals as described herein for the various embodiments.

Moreover, some embodiments may include a non-transitory computer-readable storage medium having computer-readable code stored thereon for programming a computer, server, appliance, device, processor, circuit, etc. each of which may include a processor to perform functions as described and claimed herein. Examples of such computer-readable storage mediums include, but are not limited to, a hard disk, an optical storage device, a magnetic storage device, a read-only memory (ROM), a programmable read-only memory (PROM), an erasable programmable read-only memory (EPROM), an electrically erasable programmable read-only memory (EEPROM), Flash memory, and the like. When stored in the non-transitory computer-readable medium, software can include instructions executable by a processor or device (e.g., any type of programmable circuitry or logic) that, in response to such execution, cause a processor or the device to perform a set of operations, steps, methods, processes, algorithms, functions, techniques, etc. as described herein for the various embodiments.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims. The foregoing sections include headers for various embodiments and those skilled in the art will appreciate these various embodiments may be used in combination with one another as well as individually.

What is claimed is:

1. A testing apparatus comprising:
a bottom tank comprising a plurality of coupling connectors; and
a test shaft that includes a member that is rotatably connected to the plurality of coupling connectors, a plurality of perpendicular members that extend out from the member, and a plurality of device under test (DUT) members connected to the plurality of perpendicular members, wherein the plurality of DUT members each include a plurality of posts each to support a DUT.

2. The testing apparatus of claim 1, wherein the DUT is a smart ring.

3. The testing apparatus of claim 1, wherein the DUT is a smart watch.

4. The testing apparatus of claim 1, further comprising a removable top cover located on the bottom tank.

5. The testing apparatus of claim 1, further comprising a motor connected to the member and configured to rotate the member about the plurality of coupling connectors.

6. The testing apparatus of claim 1, further comprising a motor connected to the member and configured to rotate the member about the plurality of coupling connectors in a 360-degree motion to simulate swimming motion where the posts simulate a swimmer's hands and the plurality of perpendicular members simulate the swimmer's arms.

7. The testing apparatus of claim 1, further comprising a motor connected to the member and configured to rotate the member about the plurality of coupling connectors in a 180-degree motion back and forth to simulate a walking or running motion where the posts simulate a wearer's hands and the plurality of perpendicular members simulate the wearer's arms.

8. The testing apparatus of claim 1, further comprising water in the bottom tank for simulating swimming.

9. The testing apparatus of claim 1, wherein the bottom tank includes water for simulating swimming and air for simulating walking or running.

10. The testing apparatus of claim 1, further comprising:
a motor connected to the member and configured to rotate the member about the plurality of coupling connectors; and
a control panel for controlling and configuring the motor.

11. The testing apparatus of claim 1, wherein the plurality of perpendicular members include a first perpendicular member and a second perpendicular member located about 180 degrees from the first perpendicular member.

12. The testing apparatus of claim 11, wherein the first perpendicular member includes a first DUT member and the second perpendicular member includes a second DUT member, and wherein the first DUT member and the second perpendicular member include complementary DUTs.

13. The testing apparatus of claim 12, wherein the complementary DUTs experience a same amount motion in a given test, such that each of the complementary DUTs should include similar metrics for the given test to be successful.

14. A method of testing a smart wearable device comprising steps of:
placing a plurality of smart wearable devices on a test shaft that includes (1) a member that is rotatably connected to a plurality of coupling connectors on a bottom tank, (2) a plurality of perpendicular members that extend out from the member, and (3) a plurality of device under test (DUT) members connected to the plurality of perpendicular members, wherein the plurality of DUT members each include a plurality of posts each to support a smart wearable device of the plurality of smart wearable devices; and
rotating the test shaft in the bottom tank that includes the plurality of coupling connectors.

15. The method of claim 14, wherein the rotating is in a 360-degree motion to simulate swimming motion where the posts simulate a swimmer's hands and the plurality of perpendicular members simulate the swimmer's arms.

16. The method of claim 14, wherein the rotating is in a 180-degree motion back and forth to simulate a walking or running motion where the posts simulate a wearer's hands and the plurality of perpendicular members simulate the wearer's ar.

17. The method of claim 14, wherein the bottom tank includes water for simulating swimming and air for simulating walking or running.

18. The method of claim 14, wherein the plurality of perpendicular members include a first perpendicular member and a second perpendicular member located about 180 degrees from the first perpendicular member, wherein the first perpendicular member includes a first DUT member and the second perpendicular member includes a second DUT member, and wherein the first DUT member and the second perpendicular member include complementary DUTs, and wherein the steps further include:
checking if the complementary DUTs experience a same amount motion in a given test, such that each of the complementary DUTs should include similar metrics for the given test to be successful.

19. The method of claim 14, wherein the bottom tank includes water for simulating swimming, and wherein the steps further include:
checking the plurality of smart wearable devices to determine whether they remain waterproof after a given test.

20. The method of claim 14, wherein the smart wearable device is one or more of a smart ring and a smart watch.
